(19) United States
(12) Patent Application Publication
(10) Pub. No.: US 2014/0081244 A1
(43) Pub. Date: Mar. 20, 2014

(54) PRESSURE SENSING GUIDEWIRE

(71) Applicant: Boston Scientific Scimed, Inc., Maple Grove, MN (US)

(72) Inventors: VIRGIL F. VOELLER, ST. LOUIS PARK, MN (US); ROGER N. HASTINGS, MAPLE GROVE, MN (US); BRIAN J. HANSON, SHOREVIEW, MN (US); KEVIN D. EDMUNDS, HAM LAKE, MN (US); LEONARD B. RICHARDSON, BROOKLYN PARK, MN (US); MICHAEL J. PIKUS, GOLDEN VALLEY, MN (US)

(73) Assignee: BOSTON SCIENTIFIC SCIMED, INC., MAPLE GROVE, MN (US)

(21) Appl. No.: 14/029,392

(22) Filed: Sep. 17, 2013

Related U.S. Application Data
(60) Provisional application No. 61/702,015, filed on Sep. 17, 2012.

(51) Int. Cl. A61M 25/01

(52) U.S. Cl. CPC A61M 25/01
USPC 604/528

(57) ABSTRACT

Medical devices and methods for making and using medical devices are disclosed. An example medical device includes a pressure sensing guidewire. The pressure sensing guidewire may include an elongate shaft including a core wire having a distal portion and a coil disposed over the distal portion. A pressure sensor may be disposed along the distal portion of the core wire and within the coil. One or more leads may be coupled to the pressure sensor. An opening may be formed in the coil that provides access to the pressure sensor.

### CROSS-REFERENCE TO RELATED APPLICATIONS

This application claims priority under 35 U.S.C. §119 to U.S. Provisional Application Ser. No. 61/702,015, filed Sep. 17, 2012, the entirety of which is incorporated herein by reference.

### TECHNICAL FIELD

The present disclosure pertains to medical devices, and methods for manufacturing medical devices. More particularly, the present disclosure pertains to blood pressure sensing guidewires.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example medical device includes a pressure sensing guidewire. The pressure sensing guidewire may include an elongate shaft including a core wire having a distal portion and a coil disposed over the distal portion. A pressure sensor may be disposed along the distal portion of the core wire and within the coil. One or more leads may be coupled to the pressure sensor. An opening may be formed in the coil that provides access to the pressure sensor.

Another example pressure sensing guidewire may include an elongate shaft including a core wire having a distal portion, a tubular member disposed over the distal portion of the core wire, and a distal tip coupled to a distal end of the tubular member. The tubular member may define a lumen and may have a plurality of slits formed therein. A pressure sensor may be disposed adjacent to the core wire and in fluid communication with the lumen. An opening may be formed in the tubular member. A diaphragm may extend over the opening. A pressure transmitting fluid may be disposed in the lumen that is configured to transmit pressure at the opening to the pressure sensor.

Another example pressure sensing guidewire may include an elongate shaft including a core wire having a tapered distal portion, a tubular member disposed over the tapered distal portion of the core wire, and a tip coupled to a distal end of the tubular member. The core wire and the tubular member may define electrodes of a capacitor. A lead may be attached to the tubular member and may extend proximally therefrom. The tubular member may define a lumen. A compressible fluid may be disposed within the lumen. An opening is formed in the tubular member adjacent to the distal end thereof.

Another example pressure sensing guidewire may include an elongated shaft including a core wire having a distal portion. A tube may be disposed over the distal portion. A pressure sensor disposed along the core wire and within the tube. One or more leads may be coupled to the pressure sensor. An opening may be formed in the tube that provides access to the pressure sensor.

Another example pressure sensing guidewire may include an elongate shaft including a core wire having a distal portion, a tubular member disposed over the distal portion of the core wire, and a distal tip coupled to a distal end of the tubular member. The tubular member may define a lumen and may have a plurality of slits formed therein. A pressure transmitting fluid may be disposed in the lumen. A first opening may be formed in the tubular member adjacent to the distal portion of the core wire. A first pressure sensor may be disposed adjacent to the first opening. A second opening may be formed in the tubular member adjacent to the proximal portion of the core wire. A second pressure sensor may be disposed adjacent to the second opening. An insulator may be disposed between the first pressure sensor and the second pressure sensor.

The above summary of some embodiments is not intended to describe each disclosed embodiment or every implementation of the present invention. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a side view of a portion of an example medical device;

FIG. 2A is a cross-sectional view of a portion of an example coil for use with a medical device;

FIG. 2B is a cross-sectional view of a portion of another example coil for use with a medical device;

FIG. 2C is a side view of a portion of an example medical device including the coil shown in FIG. 2B;

FIG. 3 is a partially cross-sectional side view of a portion of another example medical device;

FIG. 4 is a partially cross-sectional side view of a portion of another example medical device;

FIG. 5 is a partially cross-sectional side view of a portion of another example medical device;

FIG. 6 is a partially cross-sectional side view of the example medical device illustrated in FIG. 5 disposed in a blood vessel;

FIG. 7 is a partially cross-sectional side view of an example sensor for use with a medical device;

FIG. 8 is a partially cross-sectional side view of a portion of another example medical device;

FIG. 9 is a partially cross-sectional side view of the example medical device illustrated in FIG. 8 disposed in a blood vessel;

FIG. 10 is a partially cross-sectional side view of a portion of another example medical device;

FIG. 11 is a partially cross-sectional side view of a portion of another example medical device;

FIG. 12 is a partially cross-sectional side view of a portion of another example medical device;

FIG. 13 is a partially cross-sectional side view of portion of another example medical device and a connector;

FIG. 14 is a partially cross-sectional side view of portion of the example medical device and connector shown in FIG. 13 in an engaged configuration;

FIG. 15 is a partially cross-sectional side view of a portion of another example medical device;

FIG. 16 is a partially cross-sectional side view of a portion of another example medical device; and

FIG. 17 is a partially cross-sectional side view of a portion of another example medical device.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

During some medical interventions, it may be desirable to measure and/or monitor the blood pressure within a blood vessel. For example, some medical devices may include pressure sensors that allow a clinician to monitor blood pressure. Such devices may be useful in determining fractional flow reserve (FFR), which may be understood as the pressure after a stenosis relative to the pressure before the stenosis. A number of pressure sensing devices, however, may pose technical challenges for steering, tracking, and/or torqueing the device within the vasculature. For example, medical devices may include a relatively stiff pressure sensor located at or near the distal tip of the device and/or a relatively stiff spring tip, which may be difficult to navigate through the anatomy. Disclosed herein are a number of medical devices that include pressure sensing capabilities and may be more easily steered, tracked, and/or torqued within the anatomy.

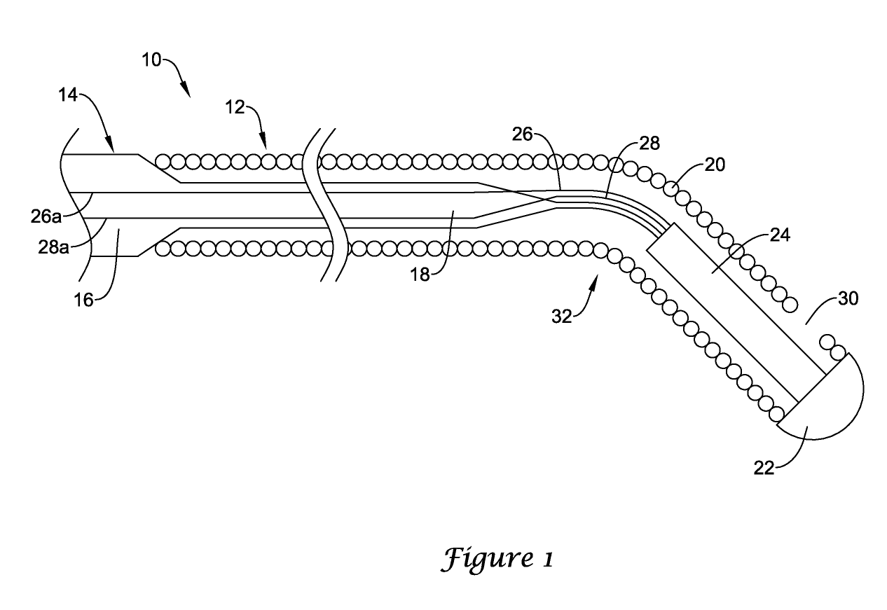

FIG. 1 illustrates a portion of an example medical device 10. In this example, medical device 10 is a blood pressure sensing guidewire 10. However, this is not intended to be limiting as other medical devices are contemplated including, for example, catheters, shafts, leads, wires, or the like. Guidewire 10 may include a guidewire shaft 12. Shaft 12 may include a core wire or member 14 having a proximal portion 16 and a distal portion 18. Distal portion 18 may be tapered or otherwise include one or more tapers and/or tapered sections. A coil 20 may be disposed about distal portion 18. A tip member 22 may be coupled to the distal end of coil 20 and define a generally atraumatic distal tip of guidewire 10.

A pressure sensor 24 may be disposed within coil 20 (e.g., at or near tip member 22). While pressure sensor 24 is shown schematically in FIG. 1, it can be appreciated that the structural form and/or type of pressure sensor 24 may vary. For example, pressure sensor 24 may include a semiconductor (e.g., silicon wafer) pressure senor, piezoelectric pressure sensor, a fiber optic or optical pressure sensor, a Fabry-Perot type pressure sensor, an ultrasound transducer and/or ultrasound pressure sensor, a magnetic pressure sensor, or the like, or any other suitable pressure sensor. To the extent applicable, any of the pressure sensors disclosed herein may be utilized in any of the medical devices disclosed herein, as appropriate.

In at least some embodiments, one or more leads, for examples leads 26/28, may be attached to pressure sensor 24 and extend proximally therefrom. A portion of leads 26/28 may be disposed within coil 20 and/or along core wire 14. Proximal portions 26a/28b of leads 26/28 may be printed on core wire 14. This may include printing leads 26/28 onto core wire 14 using ink jet or other printing technologies. Printing proximal portions 26a/28b of leads 26/28 may be desirable for a number of reasons. For example, printing proximal portions 26a/28b of leads 26/28 on core wire 14 (e.g., a solid core wire 14) may allow guidewire 10 to be manufactured without hypotubes or other structures to house or contain leads 26/28, which may simplify manufacturing.

Leads 26/28 may be appropriate for use with some types of sensors. For examples, leads 26/28 may be suitable for use with a piezoelectric pressure sensor 24. In embodiments where sensor 24 takes the form of an optical pressure sensor, a light transmitting member (e.g., a fiber optic cable, a photonic crystal, or the like) may be substituted for leads 26/28. The same may be true for other embodiments (including those disclosed herein) utilizing different types of pressure sensors. Thus, leads 26/28 may be omitted from guidewire 10 if sensor 24 takes the form of an optical pressure sensor and, instead, a fiber optic cable and/or photonic crystal may attach to sensor 24.

In at least some embodiments, an opening 30 may be formed in coil 20 that provides access for body fluids (e.g., blood) to pressure sensor 24. Opening 30 may be defined in a number of different manners. In at least some embodiments, opening 30 is defined by altering the winding pitch of coil 20 in order to define or otherwise provide spacing between adjacent windings of coil 20. Other variations in winding pitch may also be utilized for coil 20 at other regions and these variations may or may not define additional openings. In other embodiments, opening 30 may be defined by removing a portion of coil 20 in any other suitable manner.

In use, guidewire 10 may be advanced through the vasculature to a position where blood pressure monitoring is desired. When positioned as desired, blood may enter opening 30 of guidewire and come into contact with pressure sensor 24, which can sense pressure and communicate the appropriate signal along leads 26/28 to a suitable display or monitoring device (not shown). A clinician may utilize the readings from the display device to tailor the intervention to the needs of the patient or otherwise advance the goals of the intervention.

Guidewire 10 may also include a number of additional features. For example, a pre-formed bend 32 may be formed in guidewire shaft 12. In at least some embodiments, bend 32 may be positioned adjacent to pressure sensor 24 (e.g., proximal of pressure sensor 24). Bend 32 may allow guidewire 10 to be more easily navigated through the anatomy. For the purposes of this disclosure, a pre-formed bend may be understood to be a curve or bend in shaft 12 that is present when guidewire 10 is in a relaxed (e.g., un-stressed) configuration. A pre-formed bend differs from bends formed by applying a force to the shaft in order to deform or deflect the shaft.

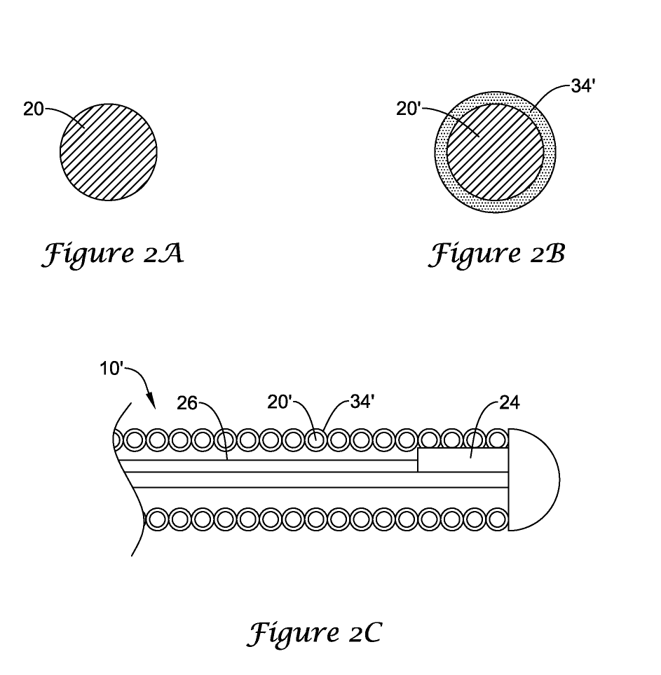

In some embodiments, coil 20 may be uncoated as shown in FIG. 2A. However, this is not intended to be limiting. For example, FIG. 2B illustrates coil 20′ (which may be used with guidewire 10) with a coating 34′. In at least some embodiments, coating 34′ may be an insulating coating. Insulated coil 20′ may be configured to function as one of the leads (e.g., be used in place of lead 26 and/or lead 28) for pressure sensor 24. For example, FIG. 2C illustrates guidewire 10′ with coil 20′ attached to pressure sensor 24. According to this embodiment, sensor 24 may still be disposed adjacent to opening 30 so that body fluids (e.g., blood) may have access to sensor 24.

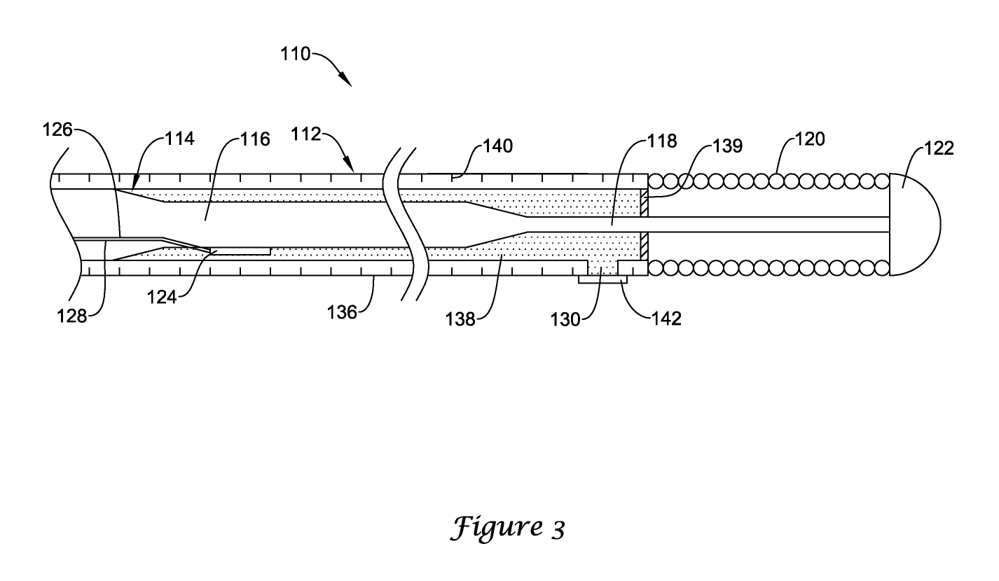

FIG. 3 illustrates another example pressure sensing guidewire 110 that may be similar in form and function to other guidewires disclosed herein. Guidewire 110 may include core wire 114 with proximal portion 116 and distal portion 118. A tubular member 136 may be coupled to core wire 114. For example, tubular member 136 may be disposed about distal portion 118. Tubular member 136 may have a plurality of slots or slits 140 formed therein. A number of different slot 140 configurations and/or arrangements are contemplated for slots/slits 140 including those disclosed herein. For example, slots 140 may extend only part way through the wall of tubular member 136. This may allow tubular member 136 to be fluid tight. Alternatively, slots 140 may extend completely through the wall of tubular member 136. In some of these later embodiments and in other embodiments, a sheath or coating (not shown) may be disposed along or within slots 140 (e.g., to substantially seal slots 140) or otherwise along the exterior of tubular member 136. Guidewire 110 may also include a distal spring tip including coil 120 and tip member 122. However, other embodiments are contemplated with differing tips and/or tip configurations.

Tubular member 136 may define a lumen and an opening 130. A membrane or diaphragm 142 may be disposed over opening 130. A pressure transmitting fluid 138 may be disposed within the lumen of tubular member 136. A variety of pressure transmitting fluids may be utilized including, for example, DOW 360 medical fluid, commercially available from Dow Corning Corporation (Midland, Mich.). The distal end of tubular member 136 may include a closed end or seal 139 so as to contain pressure transmitting fluid 138 within tubular member 136.

Pressure sensor 124 may be disposed adjacent to core wire 114 and/or tubular member 136. For example, pressure sensor 124 may be positioned along proximal portion 116 of core wire 114. This may result in pressure sensor 124 being located proximally of the more flexible portions of guidewire 110 such that pressure sensor 124 may have a smaller impact on the distal flexibility of guidewire 110. In some embodiments, a notch or cutout (not shown) may be formed in core wire 114 to house or otherwise open additional space for pressure sensor 124. Other configurations are contemplated. Leads 126/128 may be coupled to pressure sensor 124. As indicated above, leads 126/128 may be omitted or substituted with other structures, as appropriate, when the form of pressure sensor 124 varies. In general, fluid pressure may exert a force on diaphragm 142. The fluid pressure may be transferred along guidewire 110 (e.g., along tubular member 136) by pressure transmitting fluid 138 to pressure sensor 124, which can transmit a suitable signal (e.g., using any one of a variety of different signal processing techniques) to a display or other machinery.

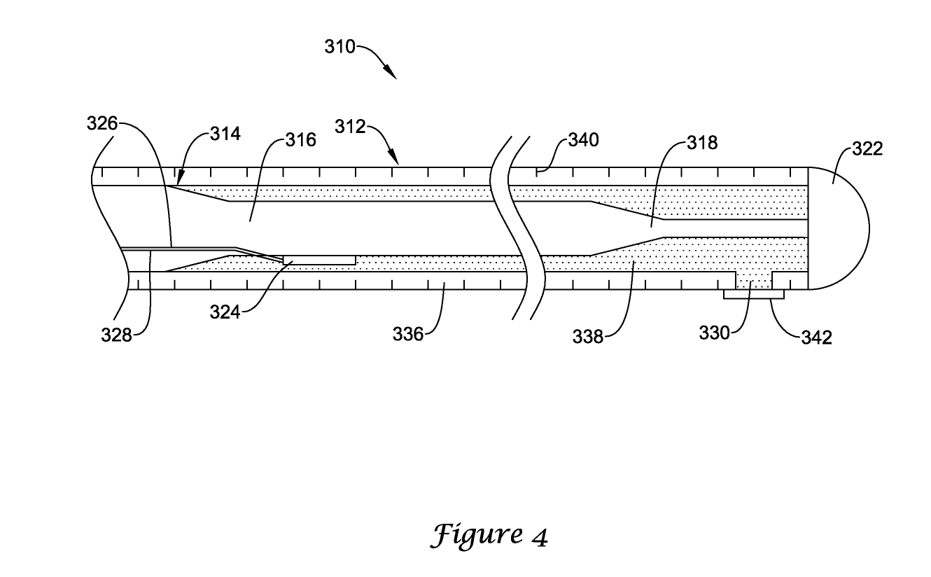

FIG. 4 illustrates another example pressure sensing guidewire 310 that may be similar in form and function to other guidewires disclosed herein. Guidewire 310 may include core wire 314 with proximal portion 316 and distal portion 318. Tubular member 336 may be coupled to core wire 314. For example, tubular member 336 may be disposed about distal portion 318. Tubular member 336 may have slots or slits 340 formed therein. Guidewire 310 may also include tip member 322 that is attached to tubular member 336.

Tubular member 336 may define a lumen and distal opening 330. Membrane or diaphragm 342 may be disposed over opening 330. Pressure sensor 324 may be disposed adjacent to core wire 314 and/or tubular member 336. Leads 326/328 may be coupled to pressure sensor 324. Pressure transmitting fluid 338 may be disposed within the lumen of tubular member 336. In general, fluid pressure may exert a force on diaphragm 342. The fluid pressure may be transferred along guidewire 310 (e.g., along tubular member 336) by pressure transmitting fluid 338 to pressure sensor 324.

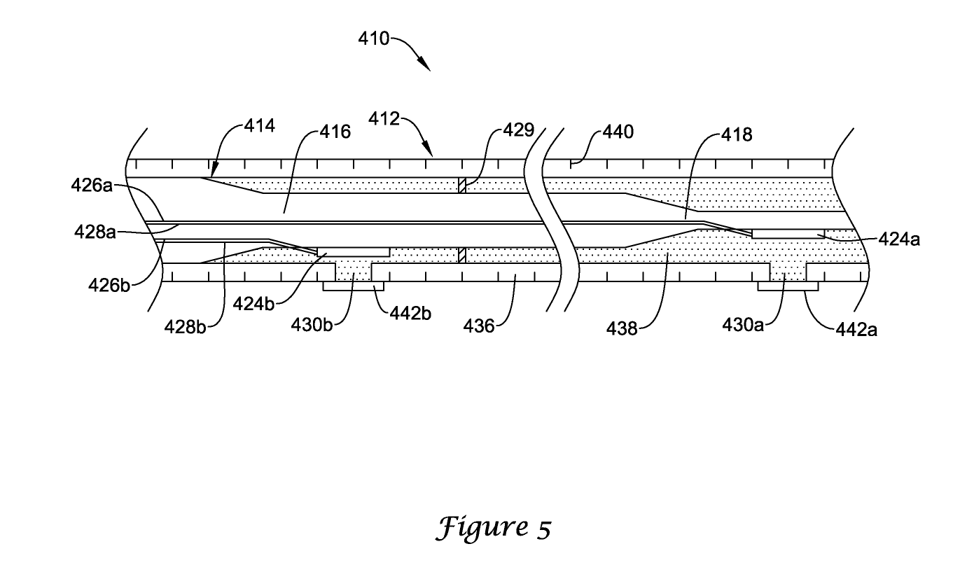

FIG. 5 illustrates another example pressure sensing guidewire 410 that may be similar in form and function to other guidewires disclosed herein. Guidewire 410 may include core wire 414 with proximal portion 416 and distal portion 418. Tubular member 436 may be coupled to core wire 414. For example, tubular member 436 may be disposed about distal portion 418. Tubular member 436 may have slots or slits 440 formed therein.

Tubular member 436 may define a lumen, a distal opening 430a, and a proximal opening 430b. A distal membrane or diaphragm 442a may be disposed over opening 430a and a proximal membrane or diaphragm 442b may be disposed over opening 430b. Alternatively, a single diaphragm may be utilized for both openings 430a/430b. Guidewire 410 may include a first pressure sensor 424a that may be disposed adjacent opening 430a and a second pressure sensor 424b that may be disposed adjacent to opening 430b. Sensors 424a/424b may be isolated from one another by a suitable fitting, O-ring, or insulator 429, which may allow sensors 424a/424b to measure pressure independently of one another. Leads 426a/428a and 426b/428b may be coupled to pressure sensors 424a/424b, respectively. Pressure transmitting fluid 438 may be disposed within the lumen of tubular member 436. In general, fluid pressure may exert a force on diaphragms 442a/44b. The fluid pressure may be transferred along guidewire 410 (e.g., along tubular member 436) by pressure transmitting fluid 438 to pressure sensors 424a/424b.

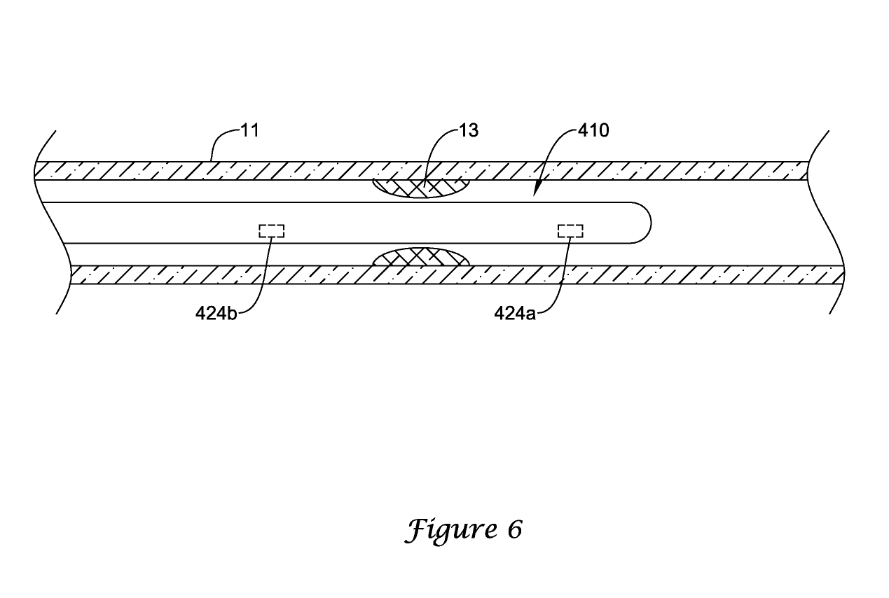

Because two sensors 424a/424b may be formed in guidewire 410, it may be possible to measure a pressure differential using sensors 424a/424b. For example, a user can advance guidewire 410 through a blood vessel 11 to a position where first sensor 424a is positioned past (e.g., distally beyond) an intravascular lesion 13 and second sensor 424b is positioned proximal of lesion 13 as shown in FIG. 6. Because the pressure at sensors 424a/424b may be measured independently of one another, a clinician may use guidewire 410 to measure or calculate FFR (e.g., the pressure after lesion 13 relative to the pressure before lesion 13). Other guidewires and devices disclosed herein may also be used to measure FFR. In addition, because a user may be able to compare the pressure on both sides of the lesion 13, guidewire 410 may be used to determine the effectiveness of a treatment on the lesion before, during, and after the intervention. This may include monitoring the pressure while advancing guidewire 410 through the blood vessel 11 until a pressure differential or drop in pressure is observed, indicating that guidewire 410 has reached and/or partially past lesion 13 as well as monitoring increases in pressure during and/or following a treatment intervention.

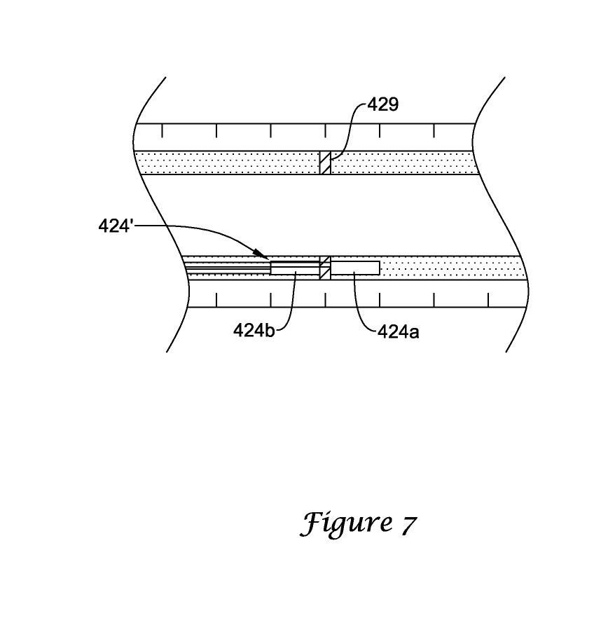

While sensors 424a/42b are shown in FIG. 6 as being distinct structures, other arrangements are contemplated. For example, FIG. 7 illustrates sensor 424′ with two independent regions or portions 424a/424b that are coupled or otherwise attached to one another. Regions 424a/424b may be positioned on either side of insulator 429. Such an arrangement would allow regions 424a/424b of sensor 424′ to independently measure pressure at different locations in a manner similar to what is disclosed herein.

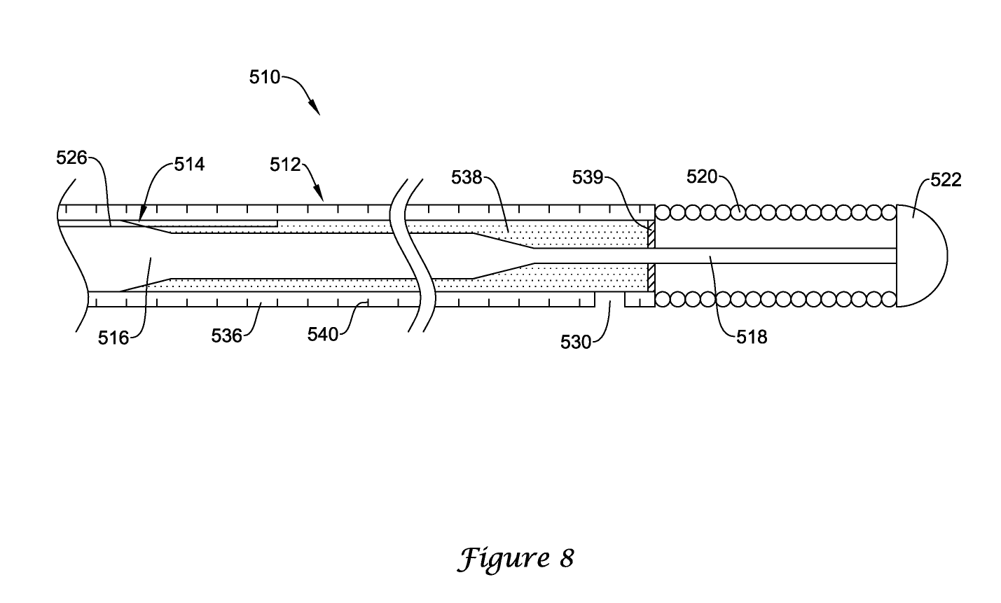

FIG. 8 illustrates another example pressure sensing guidewire 510 that may be similar in form and function to other guidewires disclosed herein. Guidewire 510 may include core wire 514 with proximal portion 516 and distal portion 518. Tubular member 536 may be coupled to core wire 514. For example, tubular member 536 may be disposed about distal portion 518. Tubular member 536 may have slots or slits 540 formed therein. Guidewire 510 may also include a distal spring tip including coil 520 and tip member 522.

Tubular member 536 may define a lumen and distal opening 530. A compressible fluid 538 may be disposed in the lumen of tubular member 536. The compressible fluid 538 may include air, carbon dioxide, saline, or the like. In at least some embodiments, surface tension may maintain compressible fluid 538 within tubular member 536 (e.g., so as to prevent compressible fluid 538 from coming out through opening 530. In other embodiments, however, tubular member 536 may have a diaphragm or membrane (not shown) disposed over opening 530 to assist in maintaining fluid 538 within tubular member 536.

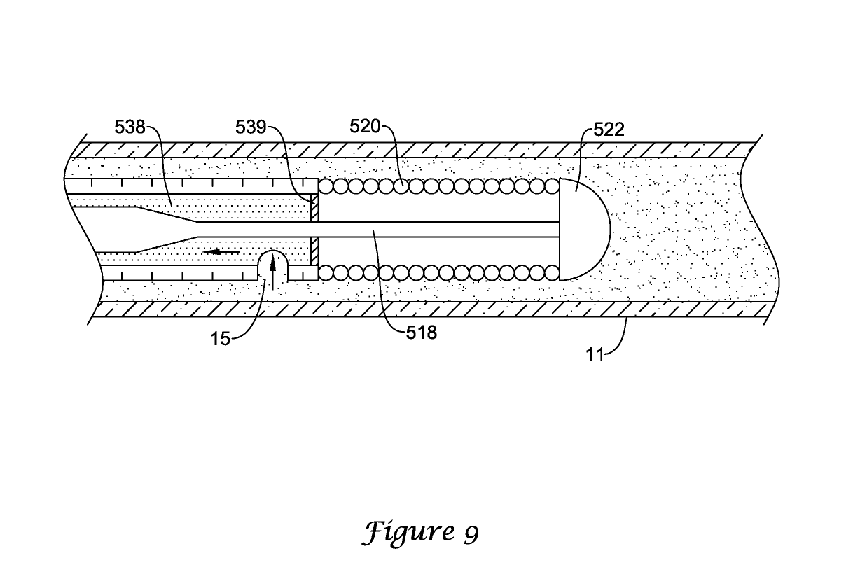

Guidewire 510, unlike other guidewires disclosed herein, may lack a separate pressure sensor or transducer and, instead, may utilize core wire 514 and tubular member 536 as the two electrodes of a coaxial capacitor. Blood 15 may act as a dielectric material such that the capacitance of the coaxial capacitor may increase as blood 15 enters the space between tubular member 536 and core wire 514 and exerts a force on compressible fluid 538 as illustrated in FIG. 9. The capacitance between core wire 514 and tubular member 536 may change (e.g., increase) as the dielectric material shifts (e.g., during systole/diastole) within guidewire 510. Accordingly, the changes in capacitance can be correlated with pressure so that guidewire 510 can be utilized to “sense” changes in pressure. In other embodiments, forces exerted on a membrane or diaphragm disposed over opening 530 (not shown) may shift compressible fluid 538 and alter the capacitance. Either way, the change in capacitance may be transmitted along guidewire 510 to a suitable display device. For example, core wire 514 may function as one of the leads for the coaxial capacitor and a secondary lead 526 may be coupled to tubular member 536. Core wire 514 and/or tubular member 536 may be electrically insulated and, for example, include an insulative coating.

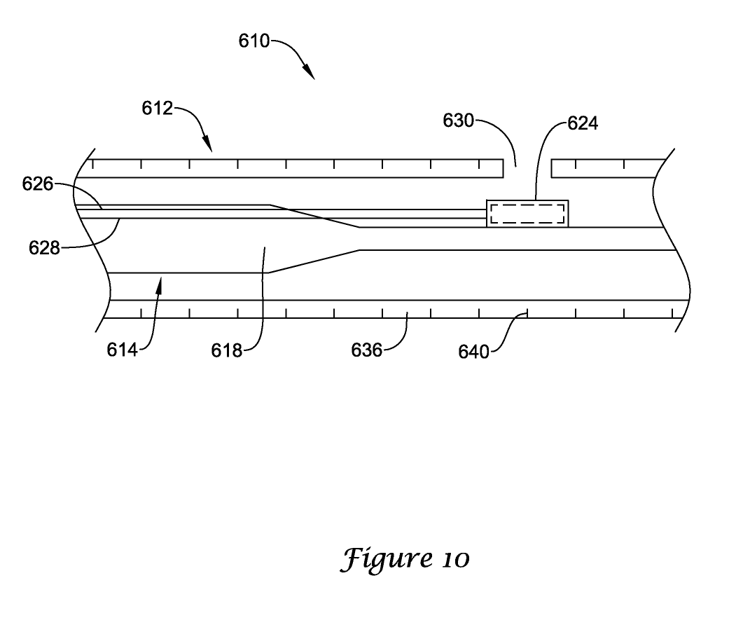

FIG. 10 illustrates another example pressure sensing guidewire 610 that may be similar in form and function to other guidewires disclosed herein. Guidewire 610 may include core wire 614 with distal portion 618. Tubular member 636 may be coupled to core wire 614. For example, tubular member 636 may be disposed about distal portion 618. Tubular member 636 may have slots or slits 640 formed therein.

Tubular member 636 may define a lumen and opening 630. Pressure sensor 624 may be disposed in the lumen and may be positioned adjacent to opening 630. Leads 626/628 may be coupled to pressure sensor 624. According to this embodiment, pressure sensor 624 may take the form of an intravascular ultrasound transducer. The ultrasound transducer 624 may be configured to contact blood entering the interior of guidewire 610 through opening 630 and measuring the pressure thereof. For example, the transducer 624 may include crystal mounted with an air or vacuum backing Flexing of the crystal under pressure may change its resonance frequency and, thus, be correlated with pressure. Alternatively, pressure sensor 624 may be piezoelectric sensor or other types of sensors disclosed herein.

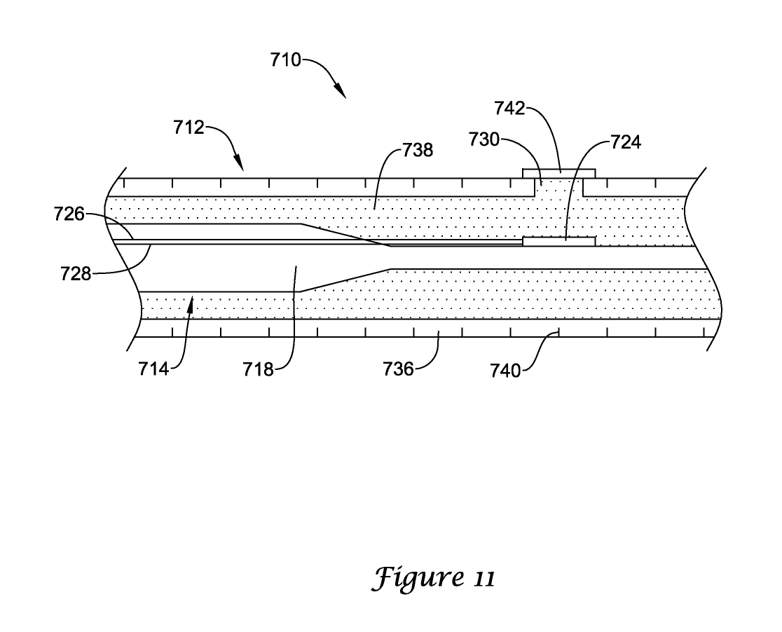

FIG. 11 illustrates another example pressure sensing guidewire 710 that may be similar in form and function to other guidewires disclosed herein. Guidewire 710 may include core wire 714 with distal portion 718. Tubular member 736 may be coupled to core wire 714. For example, tubular member 736 may be disposed about distal portion 718. Tubular member 736 may have slots or slits 740 formed therein.

Tubular member 736 may define a lumen and opening 730. Membrane or diaphragm 742 may be disposed over opening 730. Pressure sensor 724 may be disposed in the lumen and may be positioned adjacent to opening 730. Leads 726/728 may be coupled to pressure sensor 724. A fluid 738 (e.g., a fluid compatible with ultrasound such as saline) may be disposed in the lumen of tubular member 736. Much like in guidewire 610, pressure sensor 724 may take the form of an intravascular ultrasound transducer. In this embodiment, ultrasound transducer 724 may be configured to measure deflections of diaphragm 742. Accordingly, ultrasound transducer 724 may be aimed at diaphragm 742 and deflections in diaphragm 742 (e.g., in response to pressure changes) may alter (e.g., increase) the amplitude and phase of an ultrasound echo. Thus, these deflections in diaphragm 742 can be correlated with pressure.

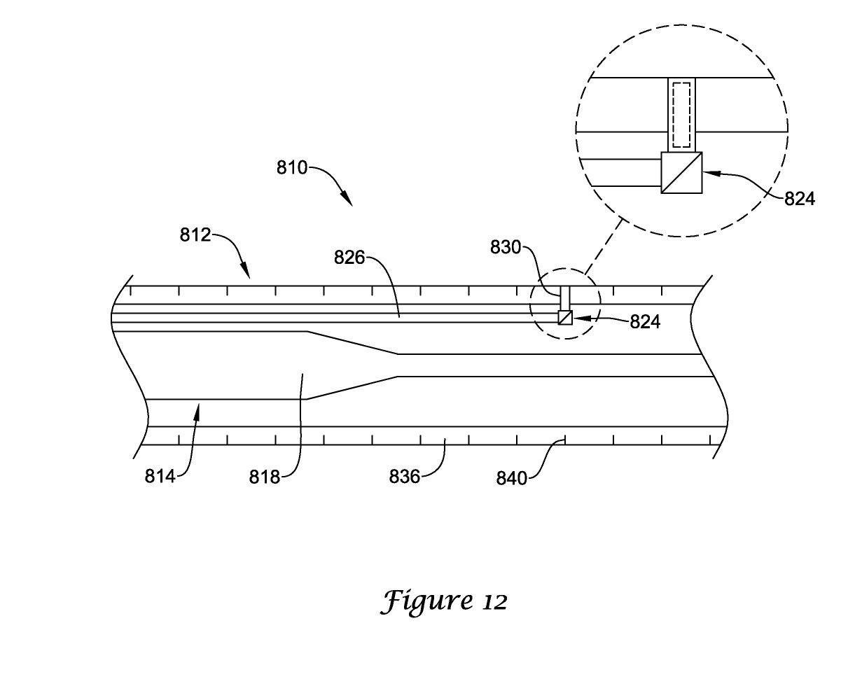

FIG. 12 illustrates another example pressure sensing guidewire 810 that may be similar in form and function to other guidewires disclosed herein. Guidewire 810 may include core wire 814 with distal portion 818. Tubular member 836 may be coupled to core wire 814. For example, tubular member 836 may be disposed about distal portion 818. Tubular member 836 may have slots or slits 840 formed therein.

Tubular member 836 may define a lumen and opening 830. Pressure sensor 824 may be disposed in the lumen and may be positioned adjacent to opening 830. According to this embodiment, pressure sensor 824 may take the form of an optical pressure sensor. A light transmitting fiber 826 may be coupled to pressure sensor 824. In at least some embodiments, fiber 826 may be a fiber optic cable. Alternatively, light transmitting fiber 826 may be a photonic crystal. The use of photonic crystal 826 may be desirable for a number of reasons. For example, in addition to being MRI compatible, a photonic crystal 826 may be an essentially “zero loss” fiber optic crystal (e.g., with essentially no loss when twisted or bent) that can transmit optical data, which can be correlated with pressure. In some embodiments, photonic crystal 826 may include one or more tapers (not shown), which may increase the flexibility of photonic crystal 826.

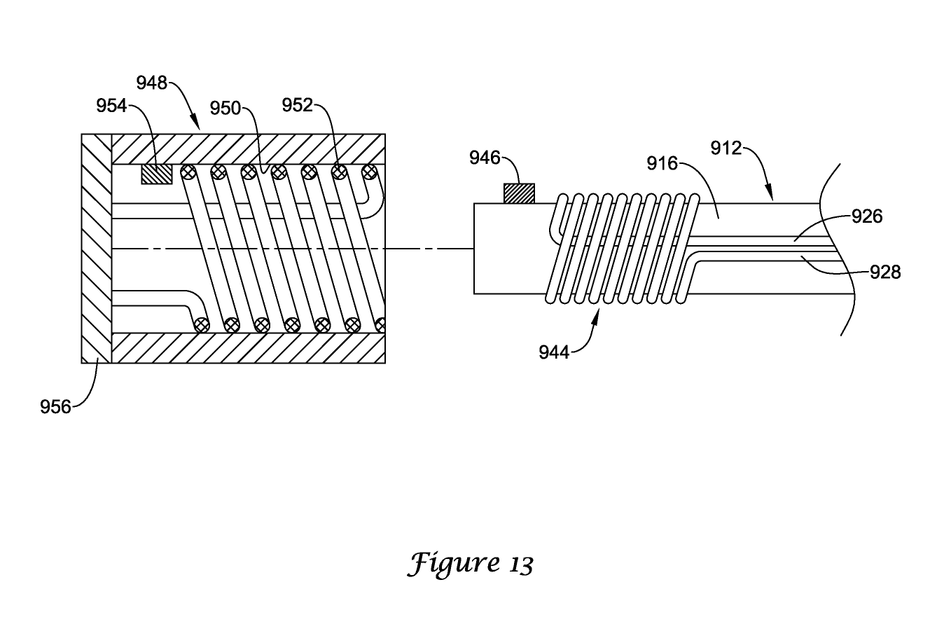

FIG. 13 is an exploded view illustrating proximal portion 916 of example guidewire shaft 912, which may be similar to other shafts disclosed herein. Here it can be seen that leads 926/928 may be disposed about proximal portion 916, for example, in a helical manner, and define a coiled region 944. A holding member 946 may be disposed on proximal portion 916. In at least some embodiment, holding member 946 may include a magnet.

Proximal portion 916 may be configured to engage a connector 948. In general, connector 948 may function as an interface between leads 926/928 and suitable electronic devices and/or displays. In general, a user may simply insert proximal portion 916 of shaft 912 into connector 948 and attach the suitable electronic devices to connector 948 (e.g., at proximal portion 956). During use of a pressure sensing guidewire such as any of those disclosed herein, a user may wish to apply torque to or otherwise rotate the guidewire shaft. When doing so, it may be desirable for electrical contact between leads 926/928 and connector 948 to be maintained. To facilitate this rotatable electrical connection, connector 948 may have an inner surface 950 having a coiled connector 952. Connector 948 may also include a holding member or magnet 954 configured to engage holding member 946 and help to securely hold proximal portion 916 of shaft 912 within connector 948. In some of these and in other embodiments, other structures may be used to securely hold proximal portion 916 of shaft 912 within connector 948 including mechanical connectors.

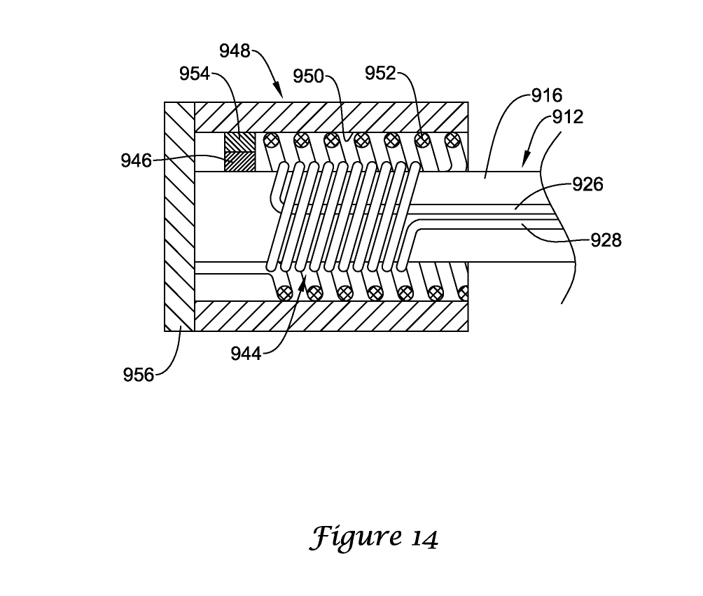

FIG. 14 illustrates proximal portion 916 of shaft 912 engaged with or otherwise coupled to connector 948. In at least some embodiments, contact between coiled connector 952 and coiled region 944 is not required. For example, an inductive coupling may be formed between coiled connector 952 and coiled region 944 where power and/or signal can be communicated therebetween while allowing for relative rotation. Such a coupling may be suitable for sensors that operate on alternating current (AC).

Alternatively, coiled connector 952 may be configured to engage coiled region 944. This may include an electrically conductive connection.

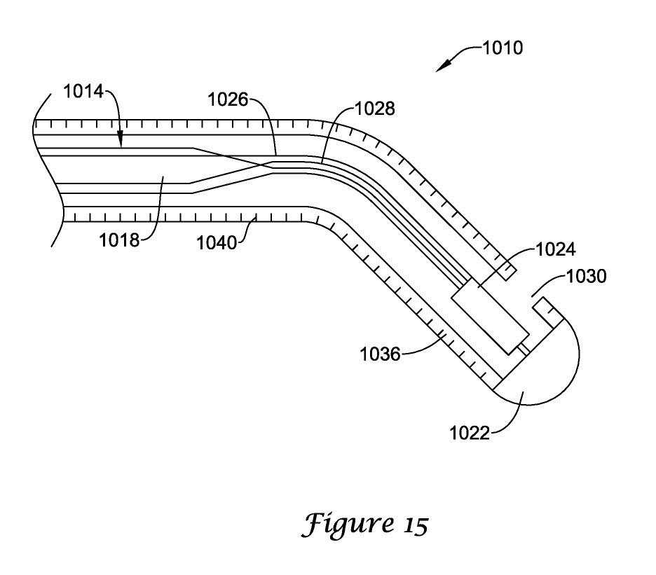

FIG. 15 illustrates another example pressure sensing guidewire 1010 that may be similar in form and function to other guidewires disclosed herein. Guidewire 1010 may include core wire 1014 with distal portion 1018. Tubular member 1036 may be coupled to core wire 1014. For example, tubular member 1036 may be disposed about distal portion 1018. Tubular member 1036 may have slots or slits 1040 formed therein. Tip member 1022 may be coupled to tubular member 1036 and/or core wire 1014.

Tubular member 1036 may define a lumen and opening 1030. Pressure sensor 1024 may be disposed in the lumen and may be positioned adjacent to opening 1030. Leads 1026/1028 may be coupled to pressure sensor 1024. According to this embodiment, fluid (e.g., blood) may enter opening 1030 and come into contact with pressure sensor 1024.

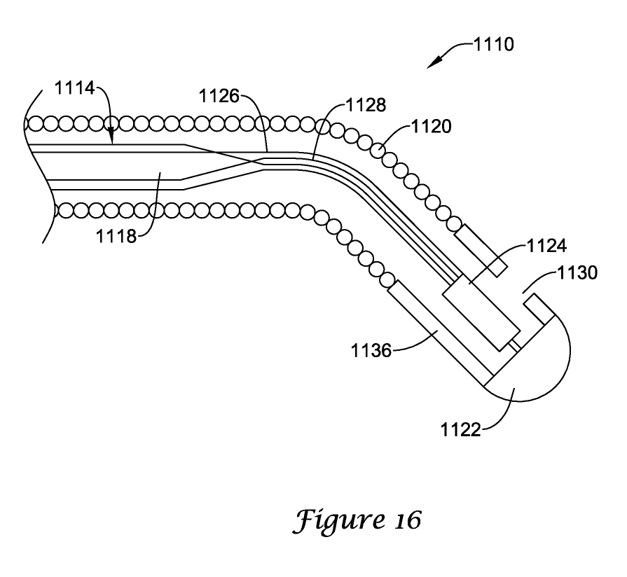

FIG. 16 illustrates another example pressure sensing guidewire 1110 that may be similar in form and function to other guidewires disclosed herein. Guidewire 1110 may include core wire 1114 with distal portion 1118. Coil 1120 may be coupled to core wire 1114. For example, coil 1120 may be disposed about distal portion 1118. Tubular member 1136 may be coupled to core wire 1114. In at least some embodiments, tubular member 1136 may be positioned at the distal end of coil 1120. Tubular member 1136 may or may not have slots or slits (not shown) formed therein. Tip member 1122 may be coupled to tubular member 1136 and/or core wire 1114.

Tubular member 1136 may define a lumen and opening 1130. Pressure sensor 1124 may be disposed in the lumen and may be positioned adjacent to opening 1130. Leads 1126/1128 may be coupled to pressure sensor 1124. According to this embodiment, fluid (e.g., blood) may enter opening 1130 and come into contact with pressure sensor 1124.

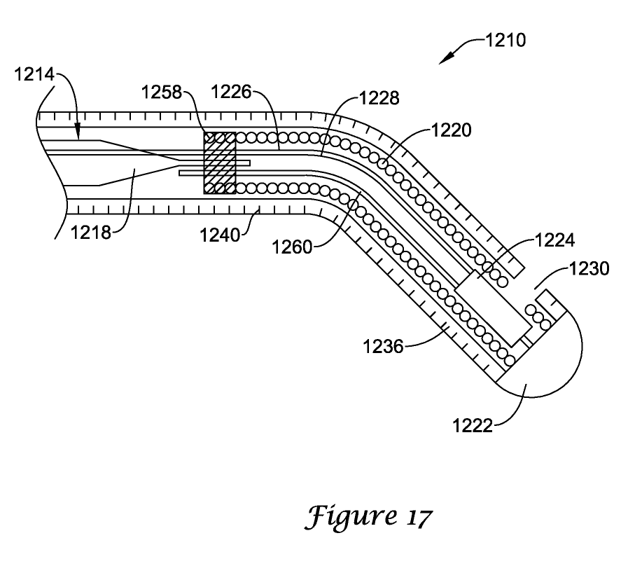

FIG. 17 illustrates another example pressure sensing guidewire 1210 that may be similar in form and function to other guidewires disclosed herein. Guidewire 1210 may include core wire 1214 with distal portion 1218. Coil 1220 may be coupled to core wire 1214. For example, coil 1220 may be disposed about distal portion 1218 and attached to core wire 1214 at a joint 1258. Joint 1258 may vary and may include a weld, an adhesive joint, a band or connector, or the like. A shaping member 1260 may also be coupled to core wire 1214 (and/or coil 1220) at joint 1258. In at least some embodiments, shaping member 1260 may include a shapeable or deformable material (e.g., linear elastic nickel-titanium alloy, stainless steel, or the like) that allows a clinician to shape (e.g., curve) a portion of guidewire 1210. Tubular member 1236 may be coupled to core wire 1214. In at least some embodiments, tubular member 1236 may be positioned over at least a portion of coil 1220. Tubular member 1236 may have slots or slits 1240 formed therein. Tip member 1222 may be coupled to tubular member 1236 and/or core wire 1214.

Tubular member 1236 may define a lumen and opening 1230. Pressure sensor 1224 may be disposed in the lumen and may be positioned adjacent to opening 1230. Leads 1226/1228 may be coupled to pressure sensor 1224. According to this embodiment, fluid (e.g., blood) may enter opening 1230 and come into contact with pressure sensor 1224.

The materials that can be used for the various components of guidewire 10 (and/or other guidewires disclosed herein) and the various tubular members disclosed herein may include those commonly associated with medical devices. For simplicity purposes, the following discussion makes reference to core wire 14 and tubular member 136 and other components of guidewires 10/110. However, this is not intended to limit the devices and methods described herein, as the discussion may be applied to other similar tubular members and/or components of tubular members or devices disclosed herein.

Core wire 14 and/or tubular member 136 may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of core wire 14 and/or tubular member 136 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of guidewire 10/110 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10/110 to achieve the same result.

In some embodiments, a degree of Magnetic Resonance Imaging (MRI) compatibility is imparted into guidewire 10/110. For example, core wire 14 and/or tubular member 136, or portions thereof, may be made of a material that does not substantially distort the image and create substantial artifacts (i.e., gaps in the image). Certain ferromagnetic materials, for example, may not be suitable because they may create artifacts in an MRI image. Core wire 14 and/or tubular member 136, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Referring now to core wire 14, the entire core wire 14 can be made of the same material along its length, or in some embodiments, can include portions or sections made of different materials. In some embodiments, the material used to construct core wire 14 is chosen to impart varying flexibility and stiffness characteristics to different portions of core wire 14. For example, proximal portion 16 and distal portion 18 of core wire 14 may be formed of different materials, for example, materials having different moduli of elasticity, resulting in a difference in flexibility. In some embodiments, the material used to construct proximal portion 16 can be relatively stiff for pushability and torqueability, and the material used to construct distal portion 18 can be relatively flexible by comparison for better lateral trackability and steerability. For example, proximal portion 16 can be formed of straightened 304v stainless steel wire or ribbon and distal portion 18 can be formed of a straightened super elastic or linear elastic alloy, for example a nickel-titanium alloy wire or ribbon.

In embodiments where different portions of core wire 14 are made of different materials, the different portions can be connected using a suitable connecting technique and/or with a connector. For example, the different portions of core wire 14 can be connected using welding (including laser welding), soldering, brazing, adhesive, or the like, or combinations thereof. These techniques can be utilized regardless of whether or not a connector is utilized. The connector may include a structure generally suitable for connecting portions of a guidewire. One example of a suitable structure includes a structure such as a hypotube or a coiled wire which has an inside diameter sized appropriately to receive and connect to the ends of the proximal portion and the distal portion. Other suitable configurations and/or structures can be utilized for the connector including those connectors described in U.S. Pat. Nos. 6,918,882 and 7,071,197 and/or in U.S. Patent Pub. No. 2006-0122537, the entire disclosures of which are herein incorporated by reference.

A sheath or covering (not shown) may be disposed over portions or all of core wire 14 and/or tubular member 136 that may define a generally smooth outer surface for guidewire 10/110. In other embodiments, however, such a sheath or covering may be absent from a portion of all of guidewire 10/110, such that core wire 14 and/or tubular member 136 and/or core wire 14 may form the outer surface. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the guidewire 10/110 (including, for example, the exterior surface of core wire 14 and/or tubular member 136) may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the sheath, or in embodiments without a sheath over portion of core wire 14 and/or tubular member 136, or other portions of guidewire 10/110. Alternatively, the sheath may comprise a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

The coating and/or sheath may be formed, for example, by coating, extrusion, co-extrusion, interrupted layer co-extrusion (ILC), or fusing several segments end-to-end.

The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present invention.

Various embodiments of arrangements and configurations of slots are also contemplated that may be used in addition to what is described above or may be used in alternate embodiments. For simplicity purposes, the following disclosure makes reference to guidewire 110, slots 140, and tubular member 136. However, it can be appreciated that these variations may also be utilized for other slots and/or tubular members. In some embodiments, at least some, if not all of slots 140 are disposed at the same or a similar angle with respect to the longitudinal axis of tubular member 136. As shown, slots 140 can be disposed at an angle that is perpendicular, or substantially perpendicular, and/or can be characterized as being disposed in a plane that is normal to the longitudinal axis of tubular member 136. However, in other embodiments, slots 140 can be disposed at an angle that is not perpendicular, and/or can be characterized as being disposed in a plane that is not normal to the longitudinal axis of tubular member 136. Additionally, a group of one or more slots 140 may be disposed at different angles relative to another group of one or more slots 140. The distribution and/or configuration of slots 140 can also include, to the extent applicable, any of those disclosed in U.S. Pat. Publication No. US 2004/0181174, the entire disclosure of which is herein incorporated by reference.

Slots 140 may be provided to enhance the flexibility of tubular member 136 while still allowing for suitable torque transmission characteristics. Slots 140 may be formed such that one or more rings and/or tube segments interconnected by one or more segments and/or beams that are formed in tubular member 136, and such tube segments and beams may include portions of tubular member 136 that remain after slots 140 are formed in the body of tubular member 136. Such an interconnected structure may act to maintain a relatively high degree of torsional stiffness, while maintaining a desired level of lateral flexibility. In some embodiments, some adjacent slots 140 can be formed such that they include portions that overlap with each other about the circumference of tubular member 136. In other embodiments, some adjacent slots 140 can be disposed such that they do not necessarily overlap with each other, but are disposed in a pattern that provides the desired degree of lateral flexibility.

Additionally, slots 140 can be arranged along the length of, or about the circumference of, tubular member 136 to achieve desired properties. For example, adjacent slots 140, or groups of slots 140, can be arranged in a symmetrical pattern, such as being disposed essentially equally on opposite sides about the circumference of tubular member 136, or can be rotated by an angle relative to each other about the axis of tubular member 136. Additionally, adjacent slots 140, or groups of slots 140, may be equally spaced along the length of tubular member 136, or can be arranged in an increasing or decreasing density pattern, or can be arranged in a non-symmetric or irregular pattern. Other characteristics, such as slot size, slot shape, and/or slot angle with respect to the longitudinal axis of tubular member 136, can also be varied along the length of tubular member 136 in order to vary the flexibility or other properties. In other embodiments, moreover, it is contemplated that the portions of the tubular member, such as a proximal section, or a distal section, or the entire tubular member 136, may not include any such slots 140.

As suggested herein, slots 140 may be formed in groups of two, three, four, five, or more slots 140, which may be located at substantially the same location along the axis of tubular member 136. Alternatively, a single slot 140 may be disposed at some or all of these locations. Within the groups of slots 140, there may be included slots 140 that are equal in size (i.e., span the same circumferential distance around tubular member 136). In some of these as well as other embodiments, at least some slots 140 in a group are unequal in size (i.e., span a different circumferential distance around tubular member 136). Longitudinally adjacent groups of slots 140 may have the same or different configurations. For example, some embodiments of tubular member 136 include slots 140 that are equal in size in a first group and then unequally sized in an adjacent group. It can be appreciated that in groups that have two slots 140 that are equal in size and are symmetrically disposed around the tube circumference, the centroid of the pair of beams (i.e., the portion of tubular member 136 remaining after slots 140 are formed therein) is coincident with the central axis of tubular member 136. Conversely, in groups that have two slots 140 that are unequal in size and whose centroids are directly opposed on the tube circumference, the centroid of the pair of beams can be offset from the central axis of tubular member 136. Some embodiments of tubular member 136 include only slot groups with centroids that are coincident with the central axis of the tubular member 136, only slot groups with centroids that are offset from the central axis of tubular member 136, or slot groups with centroids that are coincident with the central axis of tubular member 136 in a first group and offset from the central axis of tubular member 136 in another group. The amount of offset may vary depending on the depth (or length) of slots 140 and can include other suitable distances.

Slots 140 can be formed by methods such as micro-machining, saw-cutting (e.g., using a diamond grit embedded semiconductor dicing blade), electron discharge machining, grinding, milling, casting, molding, chemically etching or treating, or other known methods, and the like. In some such embodiments, the structure of the tubular member 136 is formed by cutting and/or removing portions of the tube to form slots 140. Some example embodiments of appropriate micromachining methods and other cutting methods, and structures for tubular members including slots and medical devices including tubular members are disclosed in U.S. Pat. Publication Nos. 2003/0069522 and 2004/0181174-A2; and U.S. Pat. Nos. 6,766,720; and 6,579,246, the entire disclosures of which are herein incorporated by reference. Some example embodiments of etching processes are described in U.S. Pat. No. 5,106,455, the entire disclosure of which is herein incorporated by reference. It should be noted that the methods for manufacturing guidewire 110 may include forming slots 140 tubular member 136 using these or other manufacturing steps.

In at least some embodiments, slots 140 may be formed in tubular member using a laser cutting process. The laser cutting process may include a suitable laser and/or laser cutting apparatus. For example, the laser cutting process may utilize a fiber laser. Utilizing processes like laser cutting may be desirable for a number of reasons. For example, laser cutting processes may allow tubular member 136 to be cut into a number of different cutting patterns in a precisely controlled manner. This may include variations in the slot width, ring width, beam height and/or width, etc. Furthermore, changes to the cutting pattern can be made without the need to replace the cutting instrument (e.g., blade). This may also allow smaller tubes (e.g., having a smaller outer diameter) to be used to form tubular member 136 without being limited by a minimum cutting blade size. Consequently, tubular members 20 may be fabricated for use in neurological devices or other devices where a relatively small size may be desired.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A pressure sensing guidewire, comprising:
an elongate shaft including a core wire having a distal portion and a coil disposed over the distal portion;
a pressure sensor disposed along the distal portion of the core wire and within the coil;
one or more leads coupled to the pressure sensor; and
wherein an opening is formed in the coil that provides fluid access to the pressure sensor.

2. The pressure sensing guidewire of claim 1, the opening formed in the coil is defined by a change in the pitch of the coil.

3. The pressure sensing guidewire of claim 1, wherein a distal portion of the one or more leads extend within the coil and wherein a proximal portion of the one or more leads are printed on the core wire.

4. The pressure sensing guidewire of claim 1, wherein a distal section of the elongate shaft includes a pre-formed bend.

5. The pressure sensing guidewire of claim 1, wherein the coil defines one of the one or more leads.

6. The pressure sensing guidewire of claim 5, wherein an insulator is disposed over the coil.

7. The pressure sensing guidewire of claim 1, wherein the pressure sensor includes an intravascular ultrasound transducer.

8. The pressure sensing guidewire of claim 1, wherein the pressure sensor includes a piezoelectric pressure sensor.

9. The pressure sensing guidewire of claim 1, wherein the pressure sensor includes an optical pressure sensor.

10. The pressure sensing guidewire of claim 9, wherein a fiber optic cable is coupled to the pressure sensor.

11. The pressure sensing guidewire of claim 9, wherein a photonic crystal is coupled to the pressure sensor.

12. The pressure sensing guidewire of claim 1, wherein a proximal portion of the one or more leads include a proximal coil disposed about a proximal portion of the shaft, wherein a connector is coupled to the proximal portion of the shaft, and wherein the connector includes a coil member that is configured to inductively couple with the proximal coil.

13. The pressure sensing guidewire of claim 12, wherein the connector includes a connector magnet and wherein the proximal portion of the shaft includes a proximal magnet that is configured to engage the connector magnet.

14. A pressure sensing guidewire, comprising:
an elongate shaft including a core wire having a distal portion, a tubular member disposed over the distal portion of the core wire, and a distal tip coupled to a distal end of the tubular member;
wherein the tubular member defines a lumen and has a plurality of slits formed therein;
a pressure sensor disposed adjacent to the distal portion of the core wire and in fluid communication with the lumen;
wherein an opening is formed in the tubular member;
a diaphragm extending over the opening; and
a pressure transmitting fluid disposed in the lumen that is configured to transmit pressure at the opening to the pressure sensor.

15. The pressure sensing guidewire of claim 14, wherein the pressure sensor includes an intravascular ultrasound transducer.

16. The pressure sensing guidewire of claim 14, wherein the pressure sensor includes a piezoelectric pressure sensor.

17. The pressure sensing guidewire of claim 14, wherein the pressure sensor includes an optical pressure sensor.

18. The pressure sensing guidewire of claim 17, wherein a fiber optic cable is coupled to the pressure sensor.

19. The pressure sensing guidewire of claim 17, wherein a photonic crystal is coupled to the pressure sensor.

20. A pressure sensing guidewire, comprising:
an elongate shaft including a core wire having a tapered distal portion, a tubular member disposed over the tapered distal portion of the core wire, and a tip coupled to a distal end of the tubular member;
wherein the core wire and the tubular member define electrodes of a capacitor;
a lead attached to the tubular member and extending proximally therefrom;
wherein the tubular member defines a lumen;
a compressible fluid disposed within the lumen; and
wherein an opening is formed in the tubular member adjacent to the distal end thereof.
